(19) United States
(12) Patent Application Publication
(10) Pub. No.: US 2026/0326171 A1
(43) Pub. Date: Oct. 22, 2026

(54) CONTINUOUS DISCARDED DIAPER RECYCLING SYSTEM

(71) Applicant: UTOP CO., LTD., Kaohsiung City (TW)

(72) Inventor: WEI-CHOU HSIEH, Kaohsiung City (TW)

(21) Appl. No.: 19/180,190

(22) Filed: Apr. 16, 2025

(51) Int. Cl. B09B 3/35; A61L 11/00; B03B 9/06; B09B 101/67

(52) U.S. Cl. CPC B09B 3/35; A61L 11/00; B03B 9/06; B09B 2101/67

(57) ABSTRACT

A continuous discarded diaper recycling system is disclosed, including a shredding device, a separation device, and a filtering device. The system is operable to recycle and process discarded diapers and to generate and use the processed diapers. The discarded diapers are shredded by the shredding device to form small units that are disinfected and sterilized. The small units of the shredded diapers are fed into the separation device, in which pulp, nonwoven fabrics, plastics, and polymers contained in the diapers are separated through the separation device to have pulp and polymers dissolved in water to separate from the nonwoven fabrics and plastics for subsequent filtering by the filtering device to recycle the pulp. The remaining plastics are collected for subsequent granulation to be reused.

### BACKGROUND OF THE INVENTION

### a. Technical Field of the Invention

The present invention relates to a continuous discarded diaper recycling system, and more particularly to a recycling system that recycles and processes discarded diapers and regenerates and reuses the processed diapers.

### b. Description of the Prior Art

Disposable diapers are very popular, and the group of users include infants, the elderly, and people with special needs. The disposable diapers are widely used in modern society, and this of course brings a lot of waste. According to statistics, at least 4 million pieces of discarded diapers, which are about 1,300 tons, are generated domestically every day. However, the current ways of processing diapers are incineration and landfill, which not only have a serious impact on the environment, but also consume a lot of energy, produce toxic substances, are difficult to decompose, and lead to high carbon dioxide emissions. Furthermore, since the diapers that absorb excrement have high moisture content, burning them in the incinerator will easily cause the furnace temperature to change, resulting in unstable furnace temperature and cracking of the furnace wall, which will in turn affect the service life of the incinerator. Therefore, if there are too many discarded diaper wastes, they will easily be rejected by incinerators, and this is a very thorny problem for the government and businesses.

With the advent of an aging society, the number of discarded diapers is increasing. With the increasing global awareness of environmental protection, it is imperative to actively seek solutions to processing of discarded diapers.

### SUMMARY OF THE INVENTION

The primary objective of the present invention is to provide a continuous discarded diaper recycling system, which mainly comprises a shredding device, a separation device, and a filtering device.

The shredding device receives discarded diapers and shreds the discarded diapers into a plurality of diaper small units.

The separation device comprises a material barrel, a spraying pipeline, and a receiving tray; the material barrel is rotatable by a transmission machine, and a barrel wall of the material barrel is formed with a plurality of holes having hole diameter smaller than the diaper small units, and the spraying pipeline is operable to inject water jets of a predetermined pressure into the interior of the material barrel, and the receiving tray is arranged below the material barrel.

The separation device is connected by means of a conveyor belt to the shredding device, so that the diaper small units outputted from the shredding device gets into the interior of the material barrel of the separation device.

The filtering device is generally in the form of a container and has two ends that are respectively provided with a water inlet and a water outlet, and multiple filter mesh layers are arranged between the water inlet and the water outlet, wherein the water inlet receives water draining from the receiving tray of the separation device, and the water entering the water inlet passes through the multiple filter mesh layers to be then discharged through the water outlet.

### BRIEF DESCRIPTION OF THE DRAWINGS

FIG. 1 is a schematic view illustrating a flow of the present invention; and

FIG. 2 is a schematic view illustrating a structure of the present invention.

### DETAILED DESCRIPTION OF THE PREFERRED EMBODIMENTS

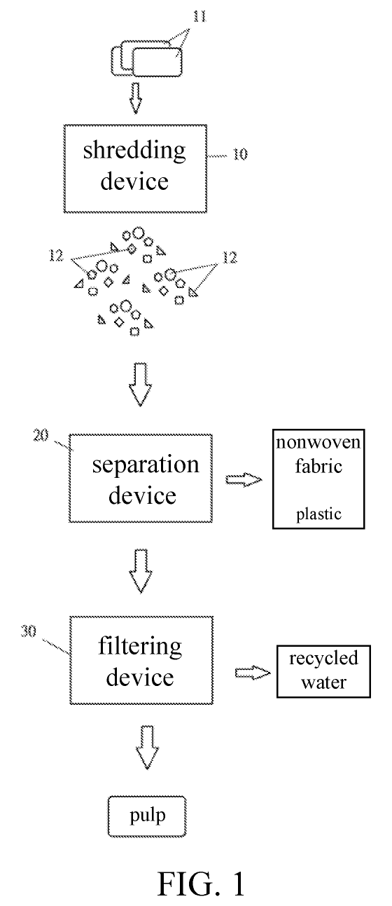

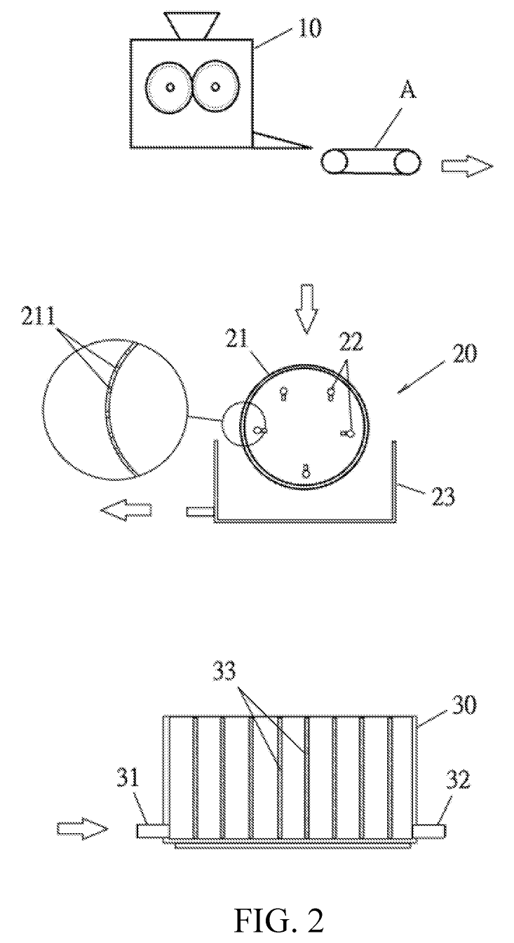

Referring to FIG. 1 which shows a flow of the present invention and FIG. 2 which shows a structure of the present invention, the present invention provides a continuous discarded diaper recycling system, which at least comprises a shredding device 10, a separation device 20, and a filtering device 30.

The shredding device 10 refers to a shearing shredding device, a roller-cutting shredding device, or a tearing shredding device. The shredding device 10 receives discarded diapers 11 and shreds the discarded diapers 11 into a plurality of diaper small units 12. The diaper small units 12 may each contain pulp, nonwoven fabrics, plastics, high-molecular polymers, and excremental waste. Further, during the course of shredding the discarded diapers 11 by the shredding device 10, a disinfectant (such as lime powder) is added in a uniform spraying manner to disinfect and sterilize the diaper small units 12.

The separation device 20 comprises a material barrel 21, a spraying pipeline 22, and a receiving tray 23. The material barrel 21 can be rotated and vibrated by a transmission machine. The receiving tray 23 is arranged below the material barrel 21. A barrel wall of the material barrel 21 is formed with a plurality of holes 211 having hole diameters smaller than each of the diaper small units 12. The spraying pipeline 22 can inject one or more water jets of a predetermined pressure into the material barrel 21. The separation device 20 is connected by means of a conveyor belt A to the shredding device 10, so that the diaper small units 12 outputted from the shredding device 10 may each get into the material barrel 21 of the separation device 20.

The filtering device 30 is generally in the form of a container and has two ends that are respectively provided with a water inlet 31 and a water outlet 32. Multiple filter mesh layers 33 are arranged between the water inlet 31 and the water outlet 32. The water inlet 31 may receive entry of liquid draining from the receiving tray 23 of the separation device 20, and the liquid entering the water inlet 31 passes through the multiple filter mesh layers 33 to then discharge from the water outlet 32. Further, the liquid discharging from the water outlet 32 is recycled by the spraying pipeline 22 of the separation device 20 for reuse.

According to the structure of the shredding device 10, the separation device 20, and the filtering device 30 of the present invention, the shredding device 10 receives the discarded diapers 11 and shreds the discarded diapers 11 into a plurality of diaper small units 12, and the diaper small units 12 are acted upon by lime powder to achieve an effect of disinfection and sterilization. At this point, the diaper small units 12 can be outputted by the conveyor belt A and conveyed into the material barrel 21 of the separation device 20. Through rotation, stirring, and vibration of the material barrel 21 and continuous injection of water from the spraying pipeline 23, the pulp, nonwoven fabric, plastic, and high-molecular polymer contained in each diaper small unit 12 can be decomposed. At this point, the pulp and high-molecular polymer are dissolved in water and flow through the holes 211 in the barrel wall of the material barrel 21 to the receiving tray 23, while the remaining non-woven fabric and plastics are retained inside the material barrel 21 due to the sizes thereof are larger than the holes 211. The plastics remaining in the material barrel 21 can be taken out for subsequent granulation, while the nonwoven fabric can be recycled using a single-shaft shredder integrated recycling machine. The pulp and the high-molecular polymer dissolved in water and flowing through the holes 211 in the barrel wall of the material barrel 21 to the receiving tray 23 are inputted through the water inlet 31 into the filtering device 30 and filtered through the multiple filter mesh layers 33 to have the pulp blocked and stopped on the surface of each of the filter mesh layers 33, so that the pulp can be subsequently taken out for secondary processing and use. The remaining water and the high-molecular polymer dissolved in the water are discharged from the water outlet 32. Further, the water discharging from the water outlet 32 is recycled and reused by the spraying pipeline 22 of the separation device 20.

In the embodiment, the discarded diapers 11 are shredded into a plurality of diaper small units 12, and pulp, nonwoven fabrics, plastics, and high-molecular polymers contained in each of the diaper small units 12 are separated one by one and taken out for recycling and reuse. Through the continuous discarded diaper recycling system according to the present invention, the thorny environmental problems caused by the discarded diapers are completely solved, the situation that the discarded diapers hinder the development of the environment is reversed, and a win-win technical model of circulation economy is created.

I claim:

1. A continuous discarded diaper recycling system, at least comprising a shredding device, a separation device, and a filtering device, wherein the shredding device is operable to receive discarded diapers and shreds the discarded diapers into a plurality of diaper small units, wherein during a course of the shredding device shredding the discarded diapers, a disinfectant is added in a uniform spraying manner to disinfect and sterilize the diaper small units; the separation device comprises the material barrel, the spraying pipeline and the receiving tray, the material barrel is rotatable by means of a transmission machine, and the receiving tray is arranged below the material barrel; a barrel wall of the material barrel of the separation device is formed with a plurality of holes having hole diameter smaller than the diaper small units, and the spraying pipeline is operable to inject a water jet of a predetermined pressure into the material barrel, and the separation device is connected through a conveyor belt to the shredding device, so that the diaper small units are outputted from the shredding device and get into the material barrel of the separation device; the filtering device is in the form of a container and has two ends that are respectively provided with a water inlet and a water outlet, and multiple filter mesh layers are arranged between the water inlet and the water outlet, wherein water outputted from the receiving tray of the separation device is receivable into the water inlet, and the water entering the water inlet is movable through the multiple filter mesh layers to discharge through the water outlet; wherein after the shredding device shreds the discarded diapers into the plurality of diaper small units, the diaper small units are conveyed by the conveyor belt to get into the material barrel of the separation device, so that through rotation and stirring by the material barrel and injection of water by the spraying pipeline, pulp, nonwoven fabrics, plastics, and high-molecular polymers contained in each of the diaper small units are decomposed such that the plastics are left in the material barrel to be subsequently recycled for use, while the pulp and the high-molecular polymers are dissolved in the water to flow into the receiving tray to be fed into the filtering device, and through filtering by the filter mesh layers, the pulp is blocked and taken out for secondary processing and use.
